(19) United States
(12) Patent Application Publication
(10) Pub. No.: US 2021/0220661 A1
(43) Pub. Date: Jul. 22, 2021

(54) Skin Tightening System

(71) Applicant: Candela Corporation, Marlborough, MA (US)

(72) Inventor: Dany Berube, Milpitas, CA (US)

(73) Assignee: Candela Corporation

(21) Appl. No.: 16/748,108

(22) Filed: Jan. 21, 2020

(51) Int. Cl. A61N 1/40; A61N 1/32; A61N 1/04; A61B 5/01

(52) U.S. Cl. CPC A61N 1/403; A61B 5/01; A61N 1/0476; A61N 1/328

(57) ABSTRACT

A skin tightening system and method features a substrate including an array of electrodes for application to a patient's skin surface. A first temperature sensor is located proximate outer electrodes of the array. A second temperature sensor is located proximate inner electrodes of the array. A first RF source powers outer electrodes of the array and a second RF source powers inner electrodes of the array. A first controller is responsive to the first temperature sensor and is configured to control the first RF source based on the temperature of the epidermis sensed by the first temperature sensor. A second controller is responsive to the second temperature sensor and is configured to control the second RF source based on the temperature of the epidermis sensed by the second temperature sensor to provide heating of the dermis.

### FIELD OF THE INVENTION

The subject invention relates to treatment systems and methods including skin tightening.

### BACKGROUND OF THE INVENTION

Various skin treatment methods are known including skin tightening using RF electrodes which apply RF energy to the dermis. In one example, a treatment hand piece includes a suction cup with a pair of electrodes therein used to treat skin urged into the suction cup. See U.S. Pat. No. 6,662,054 incorporated herein by this reference. Such a system, however, requires the operator to treat one small area for 15 to 20 minutes and then the operator must move the hand piece to treat other areas resulting in a lengthy, labor intensive procedure.

Arrays of RF electrodes have also been proposed. See U.S. Pat. No. 8,321,031 and published application nos. 2017/0136237 and 2019/013373 all incorporated herein by this reference.

### SUMMARY OF THE INVENTION

An array of electrodes in a skin tightening system is advantageous because then a larger treatment area of the dermis can be treated without the need to continually move a small hand piece to different parts of the desired treatment area. But, we have discovered that the electrodes interior to the array heat the dermis to a hotter temperature faster than the electrodes at the outer portion of the array. This is because the RF coupling between a positive electrode and any surrounding proximate negative electrodes is different as between the inner and outer electrodes. In a 5×5 array, for example, one interior positive RF electrode couples to four nearby negative electrodes resulting in a higher current density in the dermis as compared to an outer positive electrode coupling to only two or three nearby negative electrodes. As a result, for example, the dermis area beneath the inner electrodes may reach a temperature of 44° C. in 20 minutes while, in the same time period, the dermis area beneath outer electrodes may be about 2′C lower. The resulting dermis temperature profile results in a treatment area which is not treated uniformly.

In this invention, in one aspect, the temperature of the epidermis underneath the electrode array near the center thereof is measured separately from the temperature of the epidermis at the periphery of the treatment area near the outer electrodes. In this way, two controllers, each responsive to one of the temperature sensors, can independently and simultaneously automatically control two different RF sources (one for the outer electrodes and one for the inner electrodes) to achieve more uniform dermis heating across the full extent of the wide area treatment area without operator intervention.

In one example, the RF source for the inner electrodes was automatically controlled to apply a first voltage profile to the inner electrodes of the array for 20 minutes and the dermis at the area of the inner electrodes reached the temperature of 44° C. The RF source for the outer electrodes was automatically and simultaneously controlled via a second, different voltage profile for 20 minutes and the dermis at the periphery of the treatment area in the area of the outer electrodes reached almost the same temperature (in the same amount of time).

The result, in one preferred embodiment, was a more uniform heating profile, a lack of hot and cool spots, and a faster treatment without the need to move a hand piece about the treatment area or other operator intervention.

Featured is a skin tightening system. A substrate includes an array of electrodes for application to a patient's skin surface. A first temperature sensor is located proximate outer electrodes of the array and a second temperature sensor is located proximate inner electrodes of the array. A first RF source powers outer electrodes of the array and a second RF source powers inner electrodes of the array. A first controller is responsive to the first temperature sensor and is configured to control the first RF source based on the temperature of the epidermis sensed by the first temperature sensor. A second controller is responsive to the second temperature sensor and is configured to control the second RF source based on the temperature of the epidermis sensed by the second temperature sensor to provide heating of the dermis.

In one design, the array of electrodes includes alternating positive and negative electrodes. There may be one or more straps for the substrate to secure the electrodes to a patient's epidermis. Preferably, the electrode array is greater than 5×5. In one embodiment the electrodes have a diameter of X and a spacing between electrodes of at least X where X is preferably between 1-3 mm. The first and second temperatures sensors may be thermocouples attached to the substrate. The substrate may be a flexible member. Each controller is preferably a PID controller set to a target temperature. The set target temperature may be the same for each PID controller or different for each PID controller. The set target temperature may be between 42 and 45° C. In one design, each PID controller is configured to reach and maintain its set target temperature for a set application time which may be between 10 and 30 minutes.

Preferably, at least one controller is configured to calculate a thermal dose as a function of sensed temperature and to stop treatment when a predetermined thermal dose is reached for example, a predetermined thermal dose of between 1 and 10.

Also featured is a skin tightening method comprising supplying a substrate including an array of electrodes for application to a patient's skin surface, sensing a first epidermis temperature proximate outer electrodes of the array, sensing a second epidermis temperature proximate inner electrodes of the array, controlling a first RF source powering outer electrodes of the array based on the first sensed epidermis temperature, and separately controlling a second RF source powering inner electrodes of the array based on the second sensed epidermis temperature to provide heating of the dermis.

Also featured is a skin tightening method comprising applying an array of electrodes to a substrate for application to a patient's epidermis, positioning a first temperature sensor proximate outer electrodes of the array, positioning a second temperature sensor proximate inner electrodes of the array, providing a first RF source to power outer electrodes of the array, providing a second RF source to power inner electrodes of the array, configuring a first controller to be responsive to the first temperature sensor and to control the first RF source based on the temperature of the epidermis sensed by the first temperature sensor, and configuring a second controller to be responsive to the second temperature sensor and to separately control the second RF source based on the temperature of the epidermis sensed by the second temperature sensor to provide heating of the dermis.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic side cross sectional view of a prior art skin tightening hand piece;

FIG. 2 is a block diagram showing the primary component associated with a skin tightening system in accordance with an example of the invention;

FIGS. 3A and 3B are flow charts depicting the computer instructions associated with the controllers of FIG. 2 and also depicting the primary steps associated with a new method of skin tightening;

FIG. 4 is a schematic view showing a belt with first and second electrode arrays for a patient's abdomen;

FIG. 5 is a quarter-model schematic diagram showing the temperature of the inner and outer electrodes when two controllers and two RF sources were used as depicted in FIG. 2;

FIG. 6 is an exemplary graphic showing the two different voltage profiles of the two RF energy sources of FIG. 2 controlled via the flow chart of FIG. 3; and

FIG. 7 is a schematic depiction showing the temperature profile when only a single controller and RF source are used for both the inner and outer electrodes of the array.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

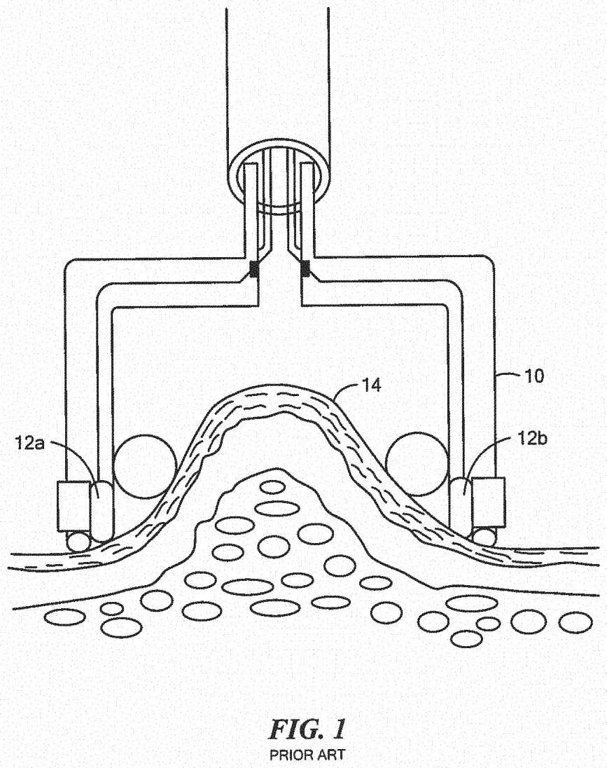

FIG. 1 shows a prior art skin tightening hand piece 10 with electrodes 12a and 12b. A vacuum pump is used to urge a region of the skin 14 to protrude into the interior of the applicator. See U.S. Pat. No. 6,662,054 incorporated herein by this reference. As noted in the Background section above, such a system requires the operator to treat one small area for 15 to 20 minutes and then the operator must move the hand piece to treat other areas resulting in a lengthy, labor intensive procedure.

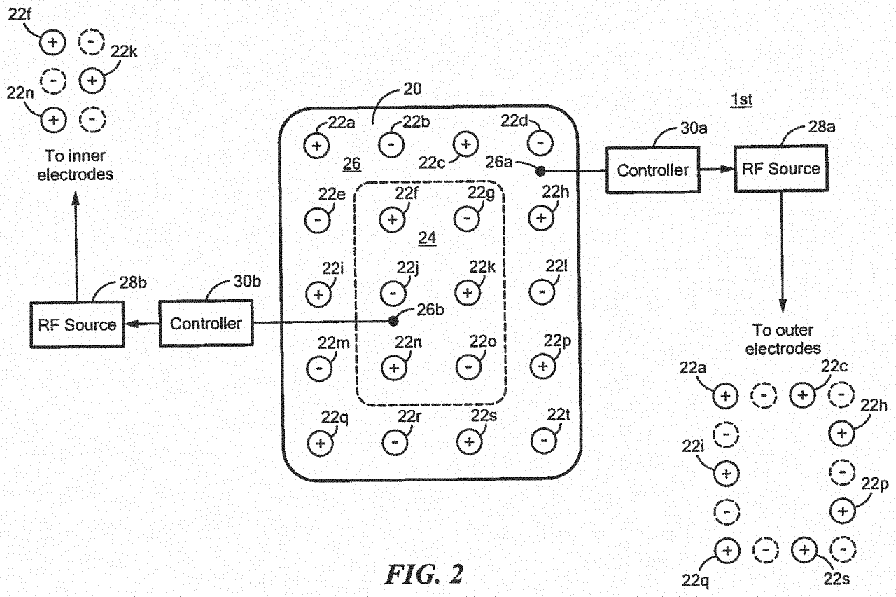

In one aspect, the new larger electrode array of FIG. 2 includes substrate 20 (typically a rigid or flexible substrate made of a dielectric medium such as Teflon, polyimide, ceramic, or other dielectric materials commonly used for printed circuitry, with alternating positive and negative electrodes 22a-22t attached thereto or integrated therewith. Typically, the array will include more electrodes, for example, an array of 5×5 to 19×19 electrodes, or more. The area of the array may typically be up to 100 cm², or more depending on the body area to be treated. The electrodes may have a diameter of between 1-3 mm and space between the electrodes may be at least 1-3 mm. The general concept is to use electrode dimensions and spacing close to the thickness of the dermis (which typically varies from 2 to 4 mm depending on the body area). Indeed, the electrical current lines usually goes as deep as the spacing between two adjacent electrodes of inverse polarities which, for biological tissue, can create a fairly uniform temperature profile throughout the target tissue to be treated—the dermis in this example. In one example, the electrodes were 2 mm in diameter and the electrode spacing was 2 mm. The electrodes are arranged to include one or more inner electrodes at region 24 and outer or peripheral electrodes at region 26. Thus, in this particular example, electrodes 22f, 22g, 22j, 22k, 22n, and 22p are “inner” electrodes and electrodes 22a, 22b, 22c, 22d, 22e, 22h, 22i, 22l, 22m, 22o, 22q, 22r, 22s, and 22t are “outer” electrodes. As a general note, the “inner” and “outer” electrodes can include more than one row.

There is a first temperature sensor 26a located proximate the outer electrodes and a second temperature sensor 26b located proximate the inner electrodes. The temperature sensors are typically in thermal contact with the skin surface, and located between two adjacent electrodes. The temperature sensors may be thermocouples or thermistors attached to substrate 20 or may be infrared temperature sensors or other temperature sensing means. At least a first radio frequency source 28a powers the positive outer electrodes at shown. Typically, all the negative outer electrodes are connected together and all the positive outer electrodes are connected together and connected to the negative and positive terminals of RF source 28a, respectively. The same is true for the inner negative electrodes and inner positive electrodes connected respectively to the negative and positive terminals of RF source 28b. The ground of the two radio frequency sources can be connected together (which is the preferred configuration due to its simplicity), or electrically isolated, for example by a transformer.

Controller 30a is responsive to temperature sensor 26a and is configured to control the first RF source 28a based on the temperature of the epidermis sensed by the first temperature sensor 26a. Controller 30b is responsive to the second temperature sensor 26b and is configured to control the second RF source 28b based on the temperature of the epidermis sensed by the second temperature sensor 26b to provide more uniform heating of the dermis.

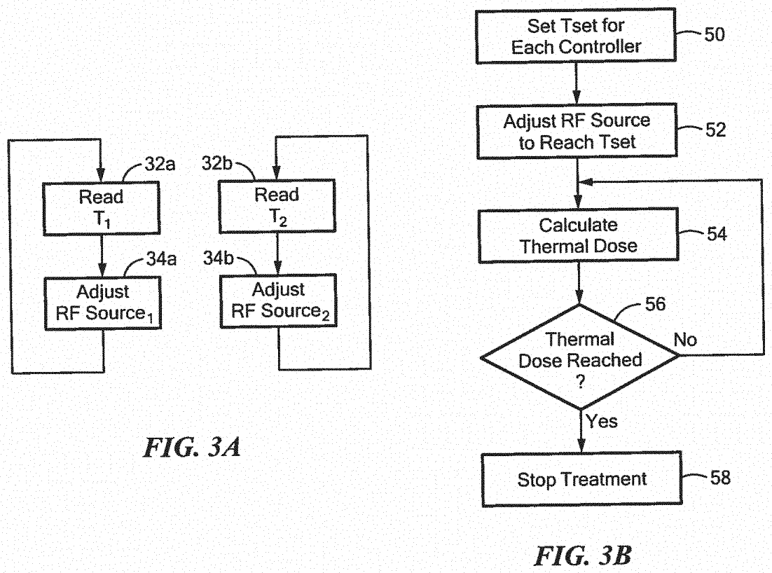

Controllers 30a, 30b may be Application Specific Integrated Circuits, microcontrollers, Proportional-Integral-Derivative controllers (PID controllers) or any suitable processor configured as disclosed herein. Typically, instructions so configured are stored in a memory and executed by a processor. These computer instructions preferably periodically read the temperature from each temperature sensor 26a, 26b, FIG. 2 as shown at steps 32a, 32b, FIG. 3A and simultaneously adjust the voltage of their respective RF sources 23a, 28b, FIG. 2 as shown at steps 34a, 34b, FIG. 3A.

In one PID embodiment, a user will select a desired target temperature (Tₛₑₜ in the equation below) step 50, FIG. 3B, and the PID will use the PID equation to calculate a control variable (usually a power, a voltage, or a current) to control its associated RF source, step 52. The desired target temperature could also be automatically selected by the system from a value or a set of values in the memory of the computer.

In one example where the voltage is the control variable, the control voltage (V) is given by the following equation:

$$V = {k}_{p} \cdot ( {T}_{set} - {T}_{measured} ) + {k}_{i} \cdot \int ( {T}_{set} - {T}_{measured} ) dt + {k}_{d} \cdot \frac{\partial ( {T}_{set} - {T}_{measured} )}{\partial t} \qquad ( 1 )$$

where kₚ, kᵢ, and k_{d} are constants (kₚ is the proportional constant, kᵢ is the integral, and k_{d} is the derivative constant), Tₛₑₜ is the set point temperature, and T_{measured} is the measured temperature measured by the temperature sensors described above. It is worthwhile noting that PID controllers can be mathematically expressed in different forms, and the concepts described in this document are broad and valid regardless of the implemented mathematical form of the PID equation.

In preferred embodiment, the PID controller is a “PI” controller where the coefficient for the derivative part of the PID equation (k_{d} in the equation above) is 0. Indeed, PI it is well known that PI controllers are less sensitive to measurement noise and therefore more stable and robust. The PID (or PI) controller is preferably adjusted by setting the constants (kₚ, kᵢ, and k_{d} in the equation above) to reach a target temperature in 5 to 10 minutes, to then maintain a target temperature for the remaining of the RF treatment. For convenient reasons to the patients and users, the total RF treatment time (or the procedure time) should be between 10 and 30 minutes. However, shorter or longer treatment sessions could be used.

One controller is associated with one RF source as shown. As a consequence, one target temperature is associated to one RF source when a PID controller is used to control the temperature. In a preferred embodiment, the target temperatures for all RF sources is the same. More specifically, target temperatures between 42 and 45° C. are useful for skin tightening procedures. However, each PID controller could be sent to a different target temperature.

Further expending the concept the skin surface temperature monitoring capabilities of the system described in this document could be used to calculate a thermal dose, which could be displayed (or not) on the GUI to provide information to the user. A thermal dose is usually calculated from the temperature measurement using the Arrhenius integral shown below.

Ω=A· [symbol] E/RT(y))δt (2)

Where A is a constant known as the “frequency factor” which represents the frequency of collisions between molecules, E is the activation energy, R is the universal gas constant, and T is the temperature (usually in Kelvins), and t is the time. Since skin tightening procedures are usually aimed at denaturing collagen in dermis, it can be useful to calculate the thermal dose received by the collagen in dermis, which is approximated by using the temperature measurements taken at the skin surface—as described in this document. For collagen, A is usually 1.14E+86 sec⁻¹, E is usually 5.62E+05 J/mol, and R is 8.314 J/mol·K. These are given as examples only and other constant values could be used. It is known in the art that partially denaturing collagen, which happens when a thermal dose between about 0.1 to about 10 has been received by a collagen-rich tissue such as dermis. Thus, the system could calculate, step 54 the thermal dose received in dermis and stop the treatment when a desired dose has been obtained, step 56-58 which, in a more specific preferred embodiment, would be between 0.3 and 5. As for the target temperature, the desired thermal dose could be selected by the user, or selected automatically by the system from a single desired thermal dose value, or from a range of desired thermal dose values stored in the memory of the controller, computer, or digital/analog memory feature(s). The value of the calculated thermal dose can be displayed on the GUI in real time, quasi real time, or after the procedure is completed using numerical form, alpha-numerical form, color range, graphs, or any other graphical forms.

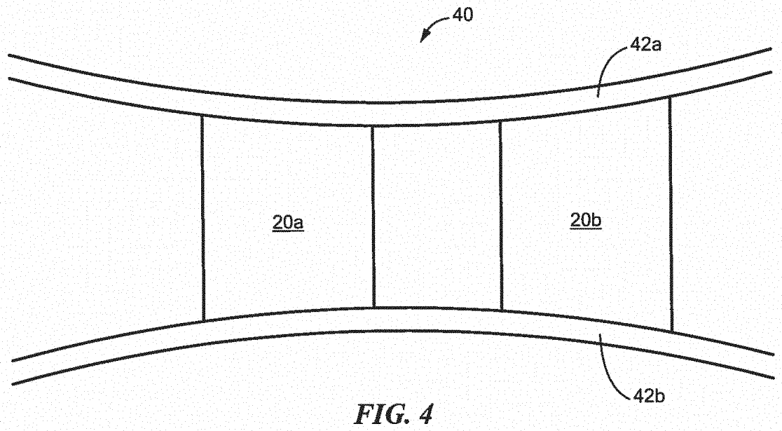

As shown in FIG. 4, a belt 40 may include two or more such substrates 20a, 20b held against a patient's abdomen via straps 42a, 42b. In general, the gap between two adjacent substrates is decreased to a minimum to avoid untreated skin in between.

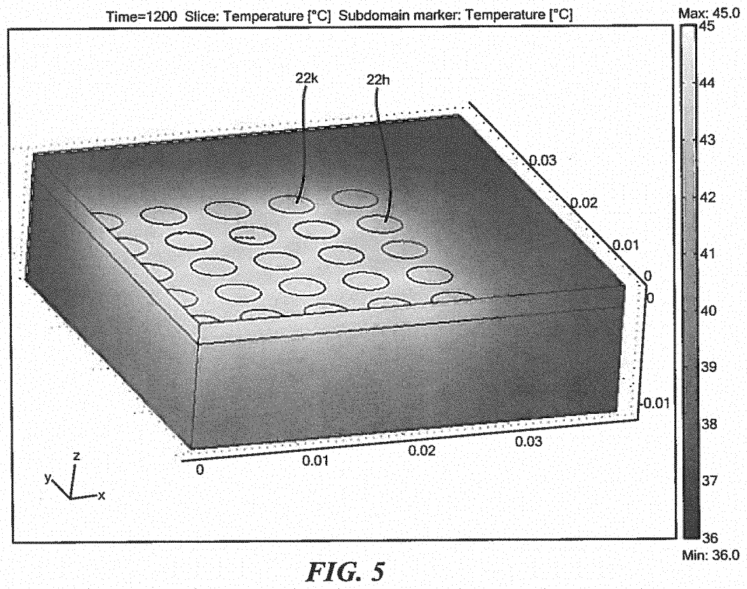

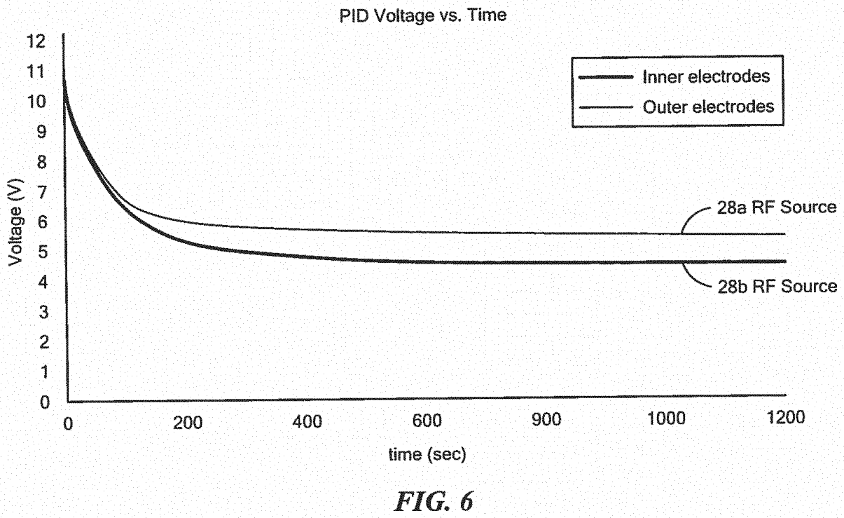

FIG. 5 shows how, in one embodiment, the target temperatures of two PID controllers 30a, 30b were set to 44° C. Skin (epidermis) tissue between two adjacent electrodes, in the vicinity of interior electrode 22k reached a temperature of about 44° C. in 20 minutes while skin tissue between two adjacent electrodes, in the vicinity of exterior peripheral electrode 22b reached almost the same temperature due to the different control algorithms of the two different controllers applying different RF power levels to the respective electrodes at shown in FIG. 6.

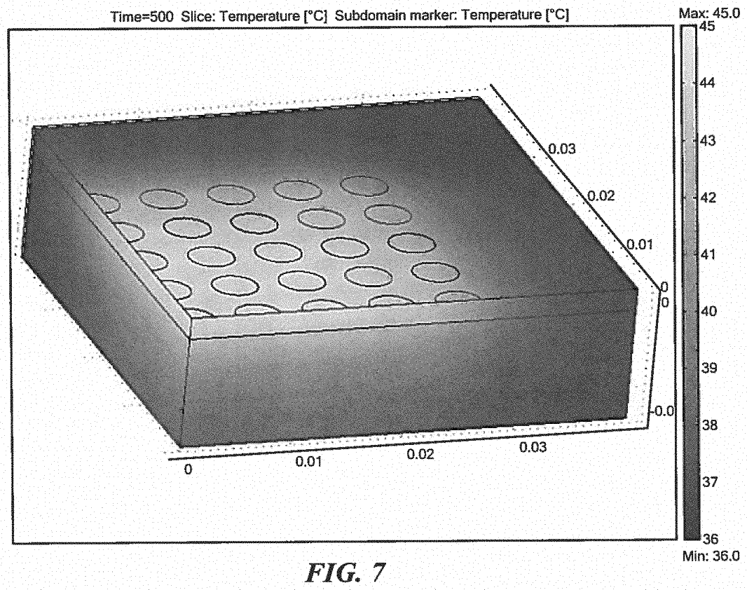

When only one temperature sensor and one controller was used as shown in FIG. 7 the resulting voltage applied to all the positive electrodes was the same and the inner electrodes reached a temperature of 44° C. in 36 minutes but the peripheral outer electrodes only reached a temperature of 42′C or less in the same time period. This difference in temperature can make a difference between an effective and less effective treatment.

The electrodes of the array may be small circular electrodes selected in size to be comparable to the dermal thickness which is about 2 mm to optimize the dermal temperature profile uniformity. A bi-polar mode may be used to take advantage of the high dermal conductivity and to keep the temperature profile in the dermis. Typically, the skin temperature is heated to a temperature of about 44° C. and maintained at that temperature for about 20 minutes during a skin tightening procedure. Typically, no skin surface cooling is necessary. Dermal and subcutaneous thicknesses are typically 2 and 10 mm, respectively. The electrode radius and the gap between two adjacent electrodes may be preferably set between 2 and 4 mm in order to allow the electrical current lines to reach the deep dermis as described earlier. The resulting voltage profile from each controller applies a voltage gradient throughout the dermis necessary to obtain uniform heating throughout the dermal space. Typically, an increased voltage is applied on the boundary or outer electrodes to compensate for the higher impedance due to the lack of ground electrodes on one side which would otherwise cause weak spots in energy deposition and non-uniform heating along the edges of the array. The voltages applied to the boundary electrodes were adjusted automatically by the PID controller to optimize the thermal profile uniformity, and to reach and maintain a specified target temperature, 44° C. in a preferred embodiment.

In one embodiment, the size or the array is approximately 5.2 by 5.2 cm. Temperature sensors may be located in the gap between any two electrodes. It is then possible to reach a prescribed target temperature in about five minutes and then to maintain the temperature for the remaining of the procedure by appropriate configuring of the PID controller.

The result, in one preferred embodiment, is a more uniform dermal temperature profile for the whole area of the patient's dermis being treated immediately underneath the electrode array. The result is a more uniform heating profile, a lack of hot and cool spots, and a faster treatment without the need to move a hand piece about the treatment area.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A skin tightening system comprising:
a substrate including an array of electrodes for application to a patient's skin surface;
a first temperature sensor proximate outer electrodes of the array;
a second temperature sensor proximate inner electrodes of the array;
a first RF source powering outer electrodes of the array;
a second RF source powering inner electrodes of the array;
a first controller, responsive to the first temperature sensor and configured to control the first RF source based on the temperature of the epidermis sensed by the first temperature sensor; and
a second controller, responsive to the second temperature sensor and configured to control the second RF source based on the temperature of the epidermis sensed by the second temperature sensor to provide heating of the dermis.

2. The system of claim 1 in which the array of electrodes includes alternating positive and negative electrodes.

3. The system of claim 1 further including one or more straps for the substrate to secure the electrodes to a patient's epidermis.

4. The system of claim 1 in which the electrode array is greater than 5×5.

5. The system of claim 1 in which the electrodes have a diameter of X and a spacing between electrodes of at least X.

6. The system of claim 5 in which X is between 1-3 mm.

7. The system of claim 1 in which the first and second temperatures sensors are attached to the substrate.

8. The system of claim 1 in which the substrate is a flexible member.

9. The system of claim 1 in which each temperature sensor is a thermocouple.

10. The system of claim 1 in which each controller is a PID controller.

11. The system of claim 10 in which each PID controller is set to a target temperature.

12. The system of claim 10 in which the set target temperature is the same for each PID controller.

13. The system of claim 10 in which the set target temperature is different for the PID controllers.

14. The system of claim 11 in which the set target temperature is between 42 and 45° C.

15. The system of claim 11 in which each PID controller is configured to reach and maintain its set target temperature.

16. The system of claim 15 in which each PID controller is configured to maintain its set target temperature for a set application time.

17. The system of claim 16 in which the set application time is between 10 and 30 minutes.

18. The system of claim 1 in which at least one controller is configured to calculate a thermal dose as a function of sensed temperature.

19. The system of claim 18 in which said at least one controller is further configured to stop treatment when a predetermined thermal dose is reached.

20. The system of claim 18 in which said at least one controller is further configured to stop treatment when a predetermined thermal dose of between 1 and 10 is reached.

21. A skin tightening method comprising:
supplying a substrate including an array of electrodes for application to a patient's skin surface;
sensing a first epidermis temperature proximate outer electrodes of the array;
sensing a second epidermis temperature proximate inner electrodes of the array;
controlling a first RF source powering outer electrodes of the array based on the first sensed epidermis temperature; and
separately controlling a second RF source powering inner electrodes of the array based on the second sensed epidermis temperature to provide heating of the dermis.

22. The method of claim 21 in which the array of electrodes includes alternating positive and negative electrodes.

23. The method of claim 21 further including one or more straps for securing the array of electrodes to a patent's abdomen.

24. The method of claim 21 in which the electrode array is greater than 5×5.

25. The method of claim 21 in which the electrodes have a diameter of X and a spacing between electrodes of at least X.

26. The method of claim 25 in which X is between 1-3 mm.

27. The method of claim 21 in which the substrate is a flexible member.

28. The method of claim 21 in which controlling the first RF source includes employing a first PID controller and controlling the second RF source includes employing a second PID controller.

29. The method of claim 28 in which each PID controller is set to a target temperature.

30. The method of claim 29 in which the set target temperature is the same for each PID controller.

31. The method of claim 29 in which the set target temperature is different for each PID controllers.

32. The method of claim 29 in which the set target temperature is between 42 and 45° C.

33. The method of claim 28 in which each PID controller is configured to reach and maintain its set target temperature.

34. The method of claim 33 in which each PID controller is configured to maintain its set target temperature for a set application time.

35. The method of claim 34 in which the set application time is between 10 and 30 minutes.

36. The method of claim 21 further including calculating a thermal dose as a function of sensed temperature.

37. The method of claim 36 further including stopping treatment when a predetermined thermal dose is reached.

38. The method of claim 37 further including stopping treatment when the predetermined thermal dose is between 1 and 10.

39. A skin tightening method comprising:
applying an array of electrodes to a substrate for application to a patient's epidermis;
positioning a first temperature sensor proximate outer electrodes of the array;
positioning a second temperature sensor proximate inner electrodes of the array;
providing a first RF source to power outer electrodes of the array;
providing a second RF source to power inner electrodes of the array;
configuring a first controller to be responsive to the first temperature sensor and to control the first RF source based on the temperature of the epidermis sensed by the first temperature sensor; and
configuring a second controller to be responsive to the second temperature sensor and to separately control the second RF source based on the temperature of the epidermis sensed by the second temperature sensor to provide heating of the dermis.

40. The method of claim 39 in which the array of electrodes includes alternating positive and negative electrodes.

41. The method of claim 39 further including supplying one or more straps for the substrate to secure the electrodes to a patient's epidermis.

42. The method of claim 39 in which the electrode array is greater than 5×5.

43. The method of claim 39 in which the electrodes have a diameter of X and a spacing between electrodes of at least X.

44. The method of claim 43 in which X is between 1-3 mm.

45. The method of claim 39 in which the first and second temperatures sensors are each a thermocouple.

46. The method of claim 39 in which the substrate is a flexible member.

47. The method of claim 39 in which each controller is a PID controller.

48. The method of claim 47 in which each PID controller is set to a target temperature.

49. The method of claim 48 in which the set target temperature is the same for each PID controller.

50. The method of claim 48 in which the set target temperature is different for the PID controllers.

51. The method of claim 47 in which the set target temperature is between 42 and 45° C.

52. The method of claim 47 in which each PID controller is configured to reach and maintain its set target temperature.

53. The method of claim 52 in which each PID controller is configured to maintain its set target temperature for a set application time.

54. The method of claim 53 in which the set application time is between 10 and 30 minutes.

55. The method of claim 39 in which at least one controller is configured to calculate a thermal dose as a function of sensed temperature.

56. The method of claim 55 in which said at least one controller is further configured to stop treatment when a predetermined thermal dose is reached.

57. The method of claim 56 in which said at least one controller is further configured to stop treatment when a predetermined thermal dose is between 1 and 10.
